(19) United States
(12) Patent Application Publication
(10) Pub. No.: US 2007/0172460 A1
(43) Pub. Date: Jul. 26, 2007

(54) Random peptide library displayed on aav vectors

(76) Inventors: Jurgen Kleinschmidt, Bammental (DE); Oliver Muller, Heidelberg (DE); Martin Trepel, Freiburg (DE); Felix Kaul, Freiburg (DE); Christoph Leder, Wendisch Even (DE)

Correspondence Address: HOWREY LLP, C/O IP DOCKETING DEPARTMENT, 2941 FAIRVIEW PARK DRIVE, SUITE 200, FALLS CHURCH, VA, 22042-2924, US

(21) Appl. No.: 10/550,374

(22) PCT Filed: Mar. 19, 2004
(86) PCT No.: PCT/EP04/02923

§ 371 Date: Jan. 12, 2007

Related U.S. Application Data
(60) Provisional application No. 60/455,594, filed on Mar. 19, 2003.

(51) Int. Cl. A61K 48/00; C40B 30/06; C40B 40/02; C40B 40/10; C07K 14/47

(52) U.S. Cl. 424/93.2; 435/5; 435/456; 530/350; 977/802

(57) ABSTRACT

Described is a method of producing a repertoire of random peptides on the surface of AAV particles wherein said random peptides are expressed as a fusion with an AAV capsid protein of an AAV particle which displays at its surface said random polypeptides. Also described is a peptide library obtainable by said method as well as a method of selecting a gene therapy vector specific for a desired cell type comprising the steps of (a) infecting the desired cell type with a peptide library of the invention and (b) harvesting AAV library particles from the supernatant and/or cell lysates. Finally, AAV vectors obtained by said method are described which are useful for gene therapy, e.g., AAV vectors targeting primary human coronary artery endothelial cells which are suitable for the treatment of diseases associated with a dysfunction of said cells.

### FIELD OF THE INVENTION

The present invention relates to a method of producing a repertoire of random peptides on the surface of AAV particles wherein said random peptides are expressed as a fusion with an AAV capsid protein of an AAV particle which displays at its surface said random polypeptides. The invention also relates to a peptide library obtainable by said method as wells as a method of selecting a gene therapy vector specific for a desired cell type comprising the steps of (a) infecting the desired cell type with a AAV display peptide library of the invention and (b) harvesting AAV library particles from the supernatant and/or cell lysates. Finally, the present invention provides AAV vectors obtained by said method which are useful for gene therapy, e.g., AAV vectors targeting preferably primary human coronary artery endothelial cells which are suitable for the treatment of diseases associated with a dysfunction of said cells.

Several documents are cited throughout the text of this specification. Each of the documents cited herein (including any manufacturer's specifications, instructions, etc.) are hereby incorporated herein by reference; however, there is no admission that any document cited is indeed prior art as to the present invention.

### BACKGROUND OF THE INVENTION

Safety and efficacy of human gene therapy continue to be the subject of considerable debate. Problems of current vectors include unintended transduction of certain tissues, adverse immune reactions, and lack of efficient transduction of the tissue of interest. The most commonly used gene transfer systems to date are derivatives of viruses. Many safety and efficacy concerns may be overcome by ablating the endogenous unspecific tropism of the vector and retargeting it to a specific tissue.

To target vectors to cell type specific receptors, ligands must be linked to the vector capsid through chemical (bispecific conjugates) or recombinant (genetically modified capsids) methods. Random phage display peptide libraries can be used to identify ligands binding to certain cell types in vitro or homing to tissue-specific endothelial receptors after intravenous injection in vivo. Such ligands have been used for therapeutic targeting in experimental models. Redirecting viral vectors by means of bispecific molecular conjugates that contain targeting peptides has substantial drawbacks for systemic treatments. These include the lack of stability of the adaptor-vector complex in vivo and immunogenicity of the adaptor molecule itself. Hence, retargeting gene therapy vectors by incorporating ligands directly into their viral capsid is preferable. On the other hand, incorporating peptides selected by phage display directly into the viral capsid can be successful but also presents limitations. The conformation of peptides in the virus protein context may be altered and the ligand-receptor binding specificity and affinity may be decreased or lost. Furthermore, a peptide isolated by phage display may not function efficiently for gene therapy vector targeting purposes if the respective receptor does not internalize the ligand or internalizes it in a manner not supporting transgene expression.

Thus, there is a need for targeted gene therapy AAV vectors, that overcome the limitations and disadvantages of the approaches of the prior art.

### SUMMARY OF THE INVENTION

The present invention relates to a method of producing a repertoire of random peptides on the surface of AAV particles wherein said random peptides are expressed as a fusion with an AAV capsid protein of an AAV particle which displays at its surface said random polypeptides. The invention also relates to a peptide library obtainable by said method as well as a method of selecting a gene therapy vector specific for a desired cell type comprising the steps of (a) infecting the desired cell type with a peptide library of the invention and (b) harvesting AAV library particles from the supernatant and/or cell lysates. Finally, the present invention provides AAV vectors obtained by said method which are useful for gene therapy, e.g., AAV vectors targeting primary human coronary artery endothelial cells which are suitable for the treatment of diseases associated with a dysfunction of said cells.

### BRIEF DESCRIPTION OF THE DRAWINGS

FIG. 1: Design and characteristics of the random peptide AAV display library construct
A: Topology of the AAV-2 capsid (Xie et al., PNAS USA 99 (2002), 10405-10410). Blue, position of the seven additional random amino-acid residues close to the top of the threefold spikes on the AAV-2 capsid surface. The insert shows a cross-section through a spike region at higher resolution with R588 shown in red and the adjacent seven amino acids deriving from the library insertion are shown in blue.
B: Part of the cap gene within the AAV-2 genome, before and after mutagenesis at nucleotide position 3967 to obtain the library backbone plasmid. The new plasmid contains two SfiI restriction sites (marked by an arrow). The two sites are separated by a “stuffer” oligonucleotide. If this stuffer is not replaced by a suitable oligonucleotide insert, the reading frame is shifted by one nucleotide, preventing generation of AAV without an insert. The nucleotide sequence is in black letters, and the respective amino acid residue sequence (single letter code) is in red letters. Blue letters indicate seven additional amino acid residues from insertion of a random oligonucleotide encoding the peptide LPQARSH. Changes compared to the wild-type sequence are highlighted in bold. The change of the wild-type capsid amino acid from N to Q was allowed as it was predicted to lead to minimal steric changes within the capsid. The insert cloned into this capsid region is framed by two new small amino acids, G and A, as the nucleotide sequences encoding those amino acids are necessary for the restriction sites and G and A serve as spacers between library peptide insert and the surrounding capsid protein.
C: Effect on the number of AAV particles generated of a seven amino acid peptide insert within the VP capsid proteins. Viruses with wild-type or mutant capsids were generated as described (pSub201 plasmid was used to produce wild-type AAV, the empty pMT187-0-3 plasmid was used as a control). Purified genome-containing AAV quantified by dot blot analysis and subsequent phosphoimager reading are represented as genomes/mL (left panel); AAV capsids quantified by A20-capsid ELISA are represented as capsids/mL (right panel). Control values were at background level. Values are means+SDs.

FIG. 2: Two-step system for production of a random AAV display peptide library from library plasmids First, AAV library transfer shuttles were generated by cotransfection of AAV genomes containing mutant cap genes flanked by ITRs and wild-type cap genes lacking ITRs in the presence of helper sequences. This led to chimeric capsids with packaged library cap genes. AAV producer cells were incubated with these transfer shuttles in the presence of helper genes at a low MOI in order to achieve a presumable uptake and propagation of one library genome per cell to ensure that the mutant capsid genome encodes for the displayed capsid protein.

FIG. 3: PCR amplification of virus DNA comprising the modified cap gene section isolated at different stages of selection DNA of AAV library pools obtained after no, 1, or 2 rounds of selection on coronary artery endothelial cells served as templates; DNA of wild-type AAV was used as a control (WT). Two PCR products were detected. The upper band corresponds to a fragment of the cap gene harboring the library oligonucleotides. The lower band corresponds to the PCR product of the wild-type cap gene. Wild-type DNA was found in the two unselected random AAV display peptide libraries 2/02-Lib and 3/02-Lib as well as in particles after the first selection round (2/02-Sel1 and 3/02-Sel1) and the second round (2/02-Sel2). The wild-type band disappeared after the second round of selection of library 3/02-Lib (3/02-Sel2), indicating that the applied AAV particles were bound and propagated more efficiently than wild-type AAV.

FIG. 4: Replication and transduction efficiencies of selected AAV clones displaying a targeting motif as shown in Table 3 in primary human coronary artery endothelial cells
A: Replicative titers in coronary artery endothelia of selected clones relative to wild-type AAV-2 or a randomly picked control clone from the initial, unselected library (LPQARSH). Values are means+SDs.
B: Transduction of primary human coronary artery endothelial cells at an MOI of 10⁴ capsids/cell with recombinant AAV vectors harboring a luciferase reporter gene packaged into modified capsids. Luciferase activities are given in relative light units [RLU] per well. Values are means+SDs. C: Transduction efficiencies of recombinant AAV vectors in HeLa cells at an MOI of 10⁴ capsids/cell. Luciferase activities are given in relative light units [RLU] per well. Values are means+SDs.

### DETAILED DESCRIPTION OF THE PRESENT INVENTION

In view of the need of improved targeted gene therapy AAV vectors, the technical problem underlying the present invention is to provide targeted gene therapy AAV-vectors, that overcome the limitations and disadvantages of the approaches of the prior art.

The solution to said technical problem is achieved by providing the embodiments characterized in the claims. For solving the above identified technical problem a peptide screening system displayed directly on the gene therapy vector itself was designed. As a tool for peptide display recombinant adeno-associated virus type 2 (AAV-2) was chosen. AAV vectors are attractive because they are non-pathogenic, transduce cells independently of proliferation, and achieve long-term transgene expression in vitro and in vivo. Heparan sulfate proteoglycan serves as a primary attachment receptor for AAV-2 (Summerford and Samulski, Journal of Virology 72 (1998), 1438-1445). Human fibroblast growth factor receptor-1 and αVβ5 integrin have been proposed as secondary receptors. The single-stranded AAV genome contains two open reading frames, rep and cap, flanked by inverted terminal repeats (ITRs) containing the cis-elements required for replication and packaging (Samulski et al., Journal of Virology 61 (1987), 3096-3101). The cap gene encodes three capsid proteins, VP1-3 (Beccera et al., Journal of Virology 62 (1988), 2745-2754). Antigenic regions of AAV-2 capsids have been mapped and their role in virus-cell interaction and infection neutralization has been described. Moreover, several sites within the AAV capsid amenable to incorporation of targeting peptides have been identified (Girod et al., Nature Medicine 5 (1999), 1052-1056; Rabinowitz et al., Virology 265 (1999), 274-285; Shi et al., Human Gene Therapy 12 (2001), 1697-1711; Wu et al., Journal of Virology 74 (2000), 8635-8647). Inserting a peptide containing an RGD-sequence into loop IV of VP1-3 leads to retargeting AAV-2 vectors to a poorly transducible integrin-expressing cell line(Girod et al., 1999). The same insertion site has been used to retarget AAV by means of phage-derived peptides (Grifman et al., Molecular Therapy 3 (2001), 964-975; Nicklin et al., Molecular Therapy 4 (2001), 174-181). The structure of the AAV-2 capsid has been resolved recently. These data may form the basis for the rational design of new ligand insertion sites (Xie et al., PNAS USA 99 (2002), 10405-10410.

Building on these studies, a peptide library displayed on the surface of AAV-2 was designed. A random peptide library was cloned into the capsid such that it was exposed on the surface of the vector at a site critical for viral attachment to the target cell, thereby diminishing the non-specific endogenous tropism of the vector. The pool of AAV particles obtained, in which each clone displays a different surface peptide, represents a random AAV display peptide library. Such libraries were used to select for AAV infecting primary human coronary artery endothelial cells. The selected particles displayed shared common peptide motifs attesting to the power of the selection. AAV vectors displaying targeting peptides showed superior transduction of coronary artery endothelial cells compared to control vectors carrying the wild-type capsid. This effect was cell type-specific because it was only observed in endothelial cells and not in HeLa control cells.

The present approach shows that viral gene therapy vectors can be used to display peptide libraries within the steric context of the intact virus capsid. Such technology allows selection for vectors transducing the cell type of interest. The AAV-construct of the present invention permits efficient directional cloning of a random oligonucleotide insert allowing the highest possible diversity of the plasmid library. The library is placed within the capsid behind arginine (R) 588 close to a position in which short peptide sequences have-been successfully introduced to retarget AAV-2. The residue R588 itself is involved in heparin binding of AAV-2 (unpublished observations) and insertions at this site have been shown to affect heparin binding activity of the AAV-2 capsid. This effect was confirmed by the observation of a reduction in heparin binding of the peptide display library as well as of the selected clones.

AAV peptide library production was developed as a three-step process (plasmid level, shuttle level, library level). A diversity of 1.1×10⁸ clones was achieved at the plasmid level. The AAV library transfer shuttle production in the next step was performed by using a vast excess of library plasmids over producer cells. This strategy resulted in more than 1012 shuttle particles with packaged library DNA, thus preserving the initial diversity of 10⁸. The final library was generated by infecting producer cells at an MOI of 1-5 replicative units per cell, resulting in a diversity of approximately 2.5×10⁷ which is limited exclusively by the number of cells used for production. Although it is impossible to determine directly the diversity of the final product, it can be expected that it comprises approximately 10⁷ different clones.

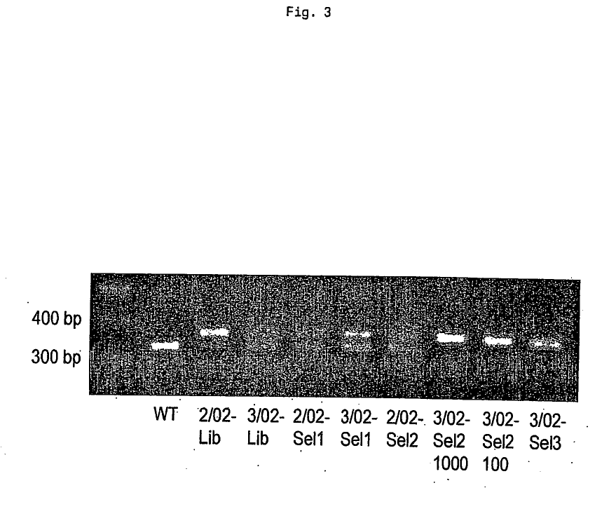

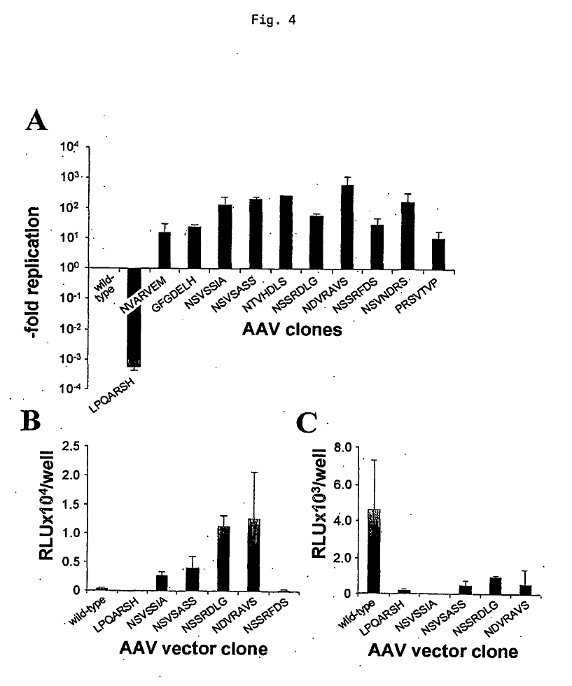

The library production method of the present invention allows packaging of wild-type AAV genomes to some degree (FIG. 3). This occurs during shuttle production and it is presumably due to homologous recombination events taking place between wild-type and library DNA. We consider wild-type DNA packaging as irrelevant as long as the complexity of the library is not diminished or the selection takes place on cells poorly susceptible to wild-type AAV infection (as is the case with the system used in this study). However, the presence of wild-type AAV may be disadvantageous for selection in cell types susceptible to AAV infection.

Preferably, for the production of the primary library (library transfer shuttles) a wild-type AAV-2 genome is used alternatively in which the codon usage of the (complete) cap gene is modified in order to prevent wild-type virus generation by homologous recombination with the library plasmids. In a further improvement of this library production step the wild-type capsid protein is generated by expression via recombinant adenovirus harbouring the codon-modified AAV-2 cap gene.

Independently produced libraries with a random peptide insert were panned on primary human coronary artery endothelial cells. After two rounds of selection, we enriched for clearly distinguishable peptide motifs mediating superior, cell-type directed infection and transduction of target cells compared with untargeted vectors in a range similar to previously reported AAV retargeting strategies (Nicklin et al., 2001). At least three peptide motifs were enriched over subsequent rounds of selection. Two of the motifs, NSVRDL^{G}/_{S} and NSVSSX^{S}/_{A} shared the tripeptide NSV, but were quite different in the other amino acid residues. Enrichment of such prominent peptide motifs after only two rounds of selection is remarkable and suggests (a) high expression of the corresponding receptors on target cells and/or (b) a clear advantage of such receptors over others in binding and/or internalizing the virus as well as allowing for viral gene expression and replication. It is important to note that almost all of the amino acid residues being part of the consensus motifs were at largely invariable positions within the peptide insert. This may indicate that the surrounding capsid protein conformation takes part in the binding of the selected clones to the target cell receptors. This fact supports our hypothesis that the selection of targeting peptides for gene therapy vectors is accomplished most appropriately by taking the capsid protein context into account during the screening process as it may be a fixed unit in conjunction with the library peptide that takes part in the targeting process.

To conclude, the above findings open new perspectives in the field of gene therapy. Any cell type of interest may be used to select for AAV vectors with altered tropism. Further development of the system will also allow for selection of AAV in vivo rendering vectors which specifically transduce certain tissues such as heart, lung or tumors. The novel vectors binding to primary human coronary artery endothelial cells described here may be applicable in clinical settings to treat human coronary heart disease. Beyond that, and perhaps more importantly, the novel technology of specific vector selection on any cell type or tissue of interest may have large potential in the development of targeted gene therapy in general.

To summarize, this is the first demonstration of a peptide display system on vector capsids of potential gene therapy vectors. The vector targeting system of the present invention has substantial advantages over other combinatorial approaches such as phage display and may have a broad range of applications in biotechnology and medicine. The present findings allow the selection of specific vector capsids to target gene therapy potentially to any given cell type of interest.

In a first embodiment, the present invention relates to a method of producing a repertoire of random peptides on the surface of AAV particles, which method comprises: expressing in a recombinant host cell nucleic acids encoding a diverse population of peptides, wherein each peptide is expressed as a fusion with an AAV capsid protein of an AAV particle which displays at its surface said diverse population of peptides, said AAV particle having the ability to replicate provided by genetic information packaged therewith and a helper vector providing the adenovirus helper functions.

Unless otherwise stated, the terms used herein are defined as described in “A multilingual glossary of biotechnological terms: (IUPAC Recommendations)”, Leuenberger, H. G. W., Nagel, B. and Kölbl, H. eds. (1995), Helvetica Chimica Acta, CH-4010 Basle, Switzerland, ISBN 3-906 390-13-6. The following definitions are provided to facilitate understanding of certain terms used throughout this specification.

The terms “treatment”, “treating” and the like are used herein to generally mean obtaining a desired pharmacological and/or physiological effect. The effect may be prophylactic in terms of completely or partially preventing a disease or symptom thereof and/or may be therapeutic in terms of partially or completely curing a disease and/or adverse effect attributed to the disease. The term “treatment” as used herein covers any treatment of a disease in a mammal, preferably a human, and includes: (a) preventing the disease from occurring in a subject which may be predisposed to the disease but has not yet been diagnosed as having it; (b) inhibiting the disease, i.e. arresting its development; or (c) relieving the disease, i.e. causing regression of the disease.

As used herein, the term “vector providing the adenovirus helper functions” relates to a vector, preferably a plasmid, which is used to complement functions which are needed for AAV replication and packaging. Examples of suitable helper vectors are shown in Example 1(A),(D).

As used herein, the term “peptide” preferably means peptides having a length of 4 to 30 amino acids. As used herein, the term “repertoire of random peptides” relates to peptides having at least 10⁴ different sequences. The random amino acid sequence is preferably encoded by random oligonucleotides with each random position being encoded by NNK or NNB codons.

Based on the experiments of the examples, below, the person skilled in the art can construct nucleic acid molecules encoding said peptides as AAV capsid fusion constructs which are presented on the AAV capsid surface in such a way that targeting of a desired cell type can be achieved. For the manipulation in prokaryotic cells by means of genetic engineering said nucleic acid molecules or parts of these sequences can be introduced into plasmids allowing a mutagenesis or a modification of a sequence by recombination of DNA sequences. By means of conventional methods (cf. Sambrook and Russel, 2001, Molecular Cloning: A Laboratory Manual, 4th edition, Cold Spring Harbor Laboratory Press, NY, USA) bases can be exchanged and natural or synthetic sequences can be added. In order to link the DNA fragments with each other adapters or linkers can be added to the fragments. Furthermore, manipulations can be performed that provide suitable cleavage sites or that remove superfluous DNA or cleavage sites. If insertions, deletions or substitutions are desired, in vitro mutagenesis, primer repair, restriction or ligation can be performed. As analysis methods usually sequence analysis, restriction analysis and other biochemical or molecular biological methods are used.

Methods for generating a pool of random oligonucleotides encoding a repertoire of peptides useful for the present invention are well known in the art, see, e.g Smith, G. P. and Scott J. K., Meth. Enzymol. 1993, 217: 228-257 and Example 1(C), below.

AAV vectors suitable for use in the present invention comprise at least the AAV-2 cap gene. AAV vectors suitable for use in the present invention are based on AAV 1 -8, with AAV-2 being preferred.

Any capsid protein of AAV is suitable for insertion of the peptides of the invention as long as their presentation on the AAV surface is not hampered. A preferred capsid protein is VP3. The preferred site of insertion is at or close to the top of the threefold spikes on the AAV-2 capsid surface with the most preferred site of insertion being at nucleotide position 3967 (behind arginine 588).

Preferably, the AAV vector is manipulated in such a way around the site for insertion of the foreign peptide that in case that no insertion by a suitable oligonucleotide occurs the reading frame is shifted by one nucleotide preventing generation of AAV vectors without an insert, as, e.g., illustrated in FIG. 1(B).

In a further embodiment, the present invention relates to a method of producing a repertoire of random peptides on the surface of AAV comprising the following steps:
(a) co-expressing in a producer host cell (i) nucleic acids encoding a diverse population of peptides, wherein each peptide is expressed as a fusion with an AAV capsid protein of an AAV particle which displays at its surface said diverse population of peptides, said AAV particle having the ability to replicate provided by genetic information packaged therewith and (ii) a helper vector providing the adenovirus helper functions and a helper plasmid containing coding sequences for a wild-type cap protein without ITRs;
(b) isolating an AAV virus library with chimeric wild-type and mutant AAV capsids (library transfer shuttles) obtained from step (a);
(c) infecting host cells with the library transfer shuttles of step (b) with a low MOI and (ii) a helper virus providing the adenovirus helper functions;
(d) harvesting and purifying the AAV capsids obtained in step (c).

In preferred embodiments of the methods of the invention, the adenovirus in step (c) is Ad5. Alternatively, any virus or genomes my be used which provide full helper functions for AAV, e.g. Ad2, HSV-1 or baculovirus.

Alternatively, a repertoire of random peptides on the surface of AAV capsids can be produced by the following steps: (a) expression in a producer host cell (i) nucleic acids encoding a diverse population of peptides, wherein each peptide is expressed as a fusion with an AAV capsid protein of an AAV particle which displays at its surface said diverse population of peptides, said AAV particle having the ability to replicate provided by genetic information packaged therewith by transfecting the nucleic acids at a concentration of approximately one plasmid per cell and (ii) a helper vector providing the adenovirus helper functions, (b) harvesting and purifying the AAV capsids obtained in step (a).

The host cells useful in the methods of the invention are not critical and comprise mammalian cells, preferably human cells such as, e.g., 911 cells, 293 cells, Hela cells, CHO cells, with 293T cells being particularly preferred.

The present invention also relates to a peptide library comprising a repertoire of random peptides on the surface of AAV particles, preferably,AAV-2 particles which is obtainable by a method of the invention.

The present invention also relates to a method of selecting a gene therapy vector specific for a desired cell type, comprising the following steps:
(a) infecting the desired cell type with a peptide library of the invention with or without superinfecting with a helpervirus for AAV production (e.g. Ad5); and
(b) harvesting AAV library particles or PCR amplified viral DNA from the supernatant and/or cell lysates. Preferably, after having harvested and titered the AAV library particles (step b), steps (a) and (b) are repeated for additional selection rounds.

Alternatively to the infection with an adenovirus, oligonucleotide inserts encoding the peptide ligands of successfully infecting AAV library particles are PCR amplified and recloned into the Sfi I site of the library plasmid pMT 187-0-3, packaged into AAV library particles as described above and used for the next round of selection.

Methods for infecting cells with AAV vectors, harvesting and purifying AAV particles (peptide library biopanning) are well known to the person skilled in the art and, e.g., described in Example 1(E), below.

After several rounds of infection and selection, viral DNA is extracted from cells productively infected with enriched AAV library particles. The oligonucleotide inserts encoding the peptide ligands are amplified by PCR and subcloned into an AAV “ITR-less” helper plasmid which is used for production of AAV vectors according to standard techniques.

By use of the methods of the invention AAV vectors targeted to any desired cell type can be selected. A preferred cell type are human coronary artery endothelial cells.

In a further embodiment, the present invention relates to a an AAV vector targeting a primary human coronary artery endothelial cell, which is obtainable by the selection method of the invention or an AAV vector targeting a primary human coronary artery endothelial cell expressing a peptide as a fusion with a capsid protein, wherein said capsid protein comprises an amino acid sequence selected from the group of amino acid sequences consisting of the amino acid sequences shown in Table 3 or 4.

Finally, the present invention relates to a method of treatment using an AAV vector selected according to a method of the present invention, e.g., an AAV vector suitable for the treatment of a disease associated with a dysfunction of primary human coronary artery endothelial cells comprising administering to a mammalian subject a therapeutically effective amount of a gene therapy AAV vector containing in its capsid protein a peptide of the invention. The invention may be applied for any disease treatable with targeted AAV vectors, e.g. infectious diseases, cardiovascular diseases or coronary heart diseases.

The pharmaceutical composition containing an AAV vector of the invention, further, contains a pharmaceutically acceptable excipient, diluent or carrier. Examples of suitable pharmaceutical carriers etc. are well known in the art and include phosphate buffered saline solutions, water, emulsions, such as oil/water emulsions, various types of wetting agents, sterile solutions etc. Such carriers can be formulated by conventional methods and can be administered to the subject at a suitable dose. Administration of the suitable compositions may be effected by different ways, e.g. by intravenous, intraperetoneal, subcutaneous, intramuscular, topical or intradermal administration. The route of administration, of course, depends, inter alia, on the kind of vector contained in the pharmaceutical composition. The dosage regimen will be determined by the attending physician and other clinical factors. As is well known in the medical arts, dosages for any one patient depends on many factors, including the patient's size, body surface area, age, sex, the particular compound to be administered, time and route of administration, the kind and stage of infection or disease, general health and other drugs being administered concurrently.

The following Examples illustrate the invention.

### EXAMPLE 1

### Materials and Methods

(A) Cell culture, transfection, virus production and titering.

Human coronary artery endothelial cells were maintained in the medium supplied by the cell provider (Promocell; Heidelberg, Germany). 293T and HeLa cells were maintained in Dulbecco's modified Eagle's medium with 10% fetal bovine serum (FBS). Transfections were performed by calcium phosphate precipitation (Hauswirth et al., Methods in Enzymology 316 (2000), 743-761). For production of AAV, we transfected 293T cells with the pTAV (Heilbronn et al., Virology 64 (1990), 3012-3018) or pSub201(Samulski et al., Journal of Virology 61 (1987), 3096-3101) plasmid, or their mutant derivatives, along with pXX6 (Xiao et al., Journal of Virology 72 (1998), 2224-2232) or pDG6VP (Dubielzig et al., Journal of Virology 73 (1999), 8989-8998), containing the adenovirus helper functions. After 72 h cells were harvested and viruses were purified by using iodixanol gradients (Hauswirth et al., 2000). Wild-type Ad5 was used for library particle amplification and inactivated thereafter at 55 ° C. for 30 min. The AAV genomic, capsid, and replicative titers were determined as described (Grimm et al., Gene Therapy 6 (1999), 1322-1330). For heparin binding, 10⁹-10¹⁰ particles in PBS containing 1 mM MgCl₂ and 2.5 mM KCl (PBS-MK) were bound to 1 mL heparin agarose (Sigma; St. Louis, Mo.), washed twice with 5 mL PBS-MK, and eluted twice with 2 mL PBS containing 1 M NaCl. Fractions were collected and analyzed with the A20-enzyme-linked immuno assay (ELISA)(Grimm et al., 1999).

(B) AAV library backbone plasmid

The plasmid serving as a backbone for cloning of the random oligonucleotides was developed from pSub201 (Samulski et al., 1987), containing the wild-type AAV genome. At nucleotide position 3967, two incompatible (to prevent autoligation after digestion) SfiI restriction sites were generated (FIG. 1b) by using the QuikChange Site-Directed Mutagenesis Kit from Stratagene (La Jolla, Calif.). First, the SfiI site surrounding nucleotide position 539 was deleted by a silent mutation using the primers 5′-GTGAGTAAGGCACCGGAGGCCC-3′ and 5′-GGGCCTCCGGTGCCTTACTCAC-3′. The two new SfiI sites including a stuffer region were generated in three mutagenesis steps, using the following primers (plus suitable complementary primers): 1) 5′-CCAACCTCCAGAGAGGCCAAAGAGGCCACAAGCAGCTACCGCAGATGTC-3′, 2) 5′-GAGGCCAAAGAGGCCAAGGCCCAAGCGGCCACCGCAGATGTCAACACAC-31, 3) 5′-CCAGAGAGGCCAGAGAGGCCAAGGCCCAGGCGGCCACCGCAG-3′. The altered DNA sequence (FIG. 1b) was verified by DNA sequencing in a clone designated pMT187-0-3.

(C) Generation of random libraries on the plasmid level

The degenerate oligonucleotide insert was designed to display two different BglI restriction sites on each end allowing for directional in-frame cloning into the SfiI digested pMT187-0-3 plasmid. The following degenerate oligonucleotide encoding a random seven residue peptide insert at position 3967 in the AAV genome was synthesized (University of Freiburg Oligonucleotide Synthesis Core Facility): 5′-CAGTCGGCCAGAGAGGC (NNK) ₇GCCCAGGCGGCTGACGAG-3′. Second strand synthesis was done by using the sequenase enzyme from the Sequenase Kit (Amersham; Freiburg, Germany) and the second strand primer 5′-CTCGTCAGCCGCCTGG-3′. The double stranded insert was purified using the QIAquick Nucleotide Removal Kit (Qiagen; Hilden, Germany). The 15 bp stuffer within pMT187-0-3 was cleaved by SfiI digestion and the plasmid was purified using the QIAquick PCR Purification Kit (Qiagen). Plasmid backbone and insert were ligated in a 1:15 molar ratio. Ligated plasmids were transformed into electrocompetent DH5α bacteria using the Gene Pulser (Biorad; Munich, Germany). Plasmid library diversity was determined by the number of clones growing from a representative aliquot of the transformed bacteria on agar plates containing 150 μg/mL ampicillin. Transformed bacteria were grown to saturation and the library plasmids were purified using Qiagen's Plasmid Prep Kit. 20 randomly picked clones from the plasmid library were sequenced to verify the presence of the random insert.

(D) Generation of AAV library transfer shuttles and the random AAV display peptide library

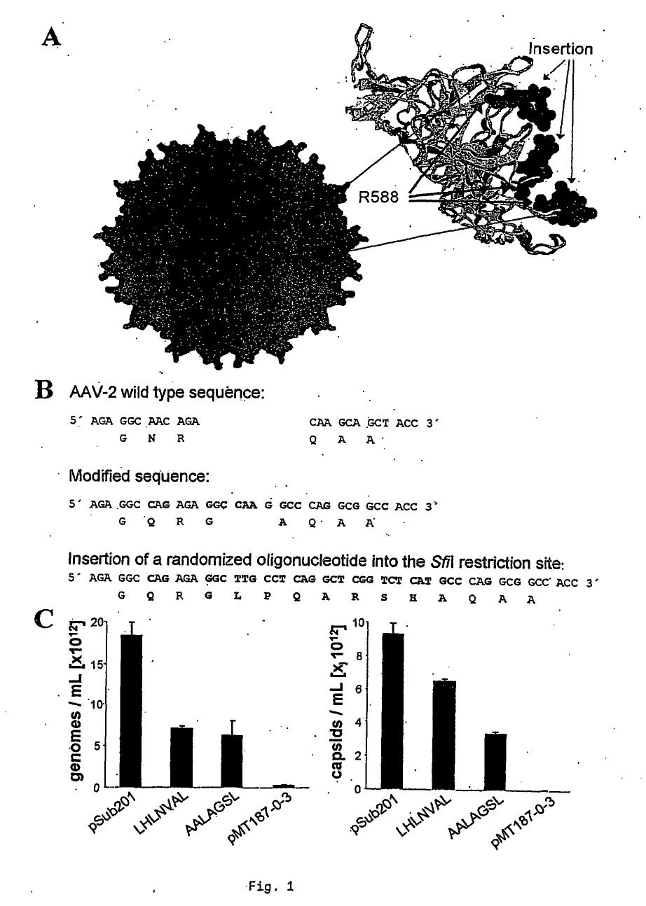

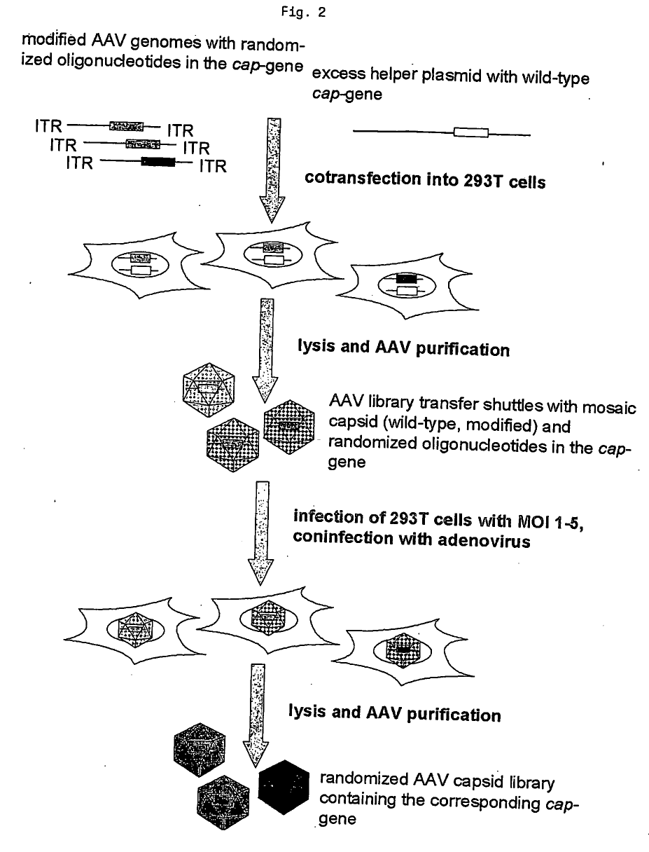

The AAV display peptide library was made from plasmids in a two-step system (FIG. 2). First, the AAV library genomes were packaged into chimeric wild-type and mutant AAV capsids (“AAV library transfer shuttles”). Then, 293T cells were transfected using a 1:1:2 ratio of the pXX2 plasmid (containing the wild-type cap gene without ITRs) (Xiao et al., 1998) and the library plasmids along with the pXX6 helper plasmid (Xiao et al., 1998). The resulting AAV library transfer shuttles were harvested, purified, and titered. The random AAV display peptide library was obtained by infection of 293T cells with the AAV library transfer shuttles at an MOI of 1 and 5 replicative units per cell and superinfection with Ad5 at an MOI of 2.5 plaque-forming units (pfu)/cell. Cells were harvested after 36 h.

(E) AAV peptide library biopanning

1.2×10⁶ primary human coronary artery endothelial cells were infected at 70% confluency with the AAV display peptide library at an MOI of 100 or 400 capsids/cell, respectively. After 6 h cells were washed with PBS followed by incubation with Ad5 at an MOI of 10 pfu/cell until observing 50% cytopathic effect. Replicated AAV particles were harvested from supernatant and from cell lysates and titered. For each subsequent selection round, preselected AAV library particles were reapplied to the target cells at an MOI of 100 to 1000 capsids/cell.

(F) PCR and Sequencing of AAV library clones

DNA extracted from AAV by the QIAamp Tissue Kit (Qiagen) served as template for a PCR with the primers 5′-GGTTCTCATCTTTGGGAAGCAAG-3′ and 5′-TGATGAGAATCTGTGGAGGAG-3′. PCR products were analyzed by gel electrophoresis, digested with BglI and cloned into the SfiI-digested pMT187-0-3 plasmid. Randomly assigned clones were sequenced using the primer 5′-GTGTGTTGACATCTGCGGTAGCTGGTGCTGTGTTGCCTCTCTGGAGGTTGGTAG-3′.

(G) Single AAV clone replication assay

AAV clones harboring modified capsids were generated using pMT187-0-3 plasmid containing PCR amplified inserts (c.f. F) and titered. Replicative titers were determined in primary coronary artery endothelial cells grown in 96-well plates. 3000 cells/well were incubated with serial dilutions of AAV clones, superinfected with Ad5 at an MOI of 10 pfu/cell and processed as described (Grimm et al., 1999).

(H) Single AAV clone gene transfer assay

To obtain rAAV-vectors carrying a luciferase reporter gene, the modified capsid region was excised by digestion with XcmI/BsiWI from plasmids of selected AAV clones and inserted into pBSΔTR18 (Weger et al., 1997), providing rep- and cap-genes without ITRs. 293T cells were cotransfected with the modified pBSΔTR18, pDGΔVP, and pUF2CMV-Luc, a derivative of pUF2 (Zolotukhin et al., 1996) harboring the luciferase gene from pGL3-basic (Promega, Mannheim, Germany). 3000 coronary endothelial cells per well and 5000 HeLa cells per well were seeded in 96-well plates and incubated with AAV luciferase vectors at an MOI of 10⁴ capsids/cell. After four days, cells were harvested and reporter gene activity was determined by the firefly luciferase assay (Promega).

### EXAMPLE 2

### Generation and Evaluation of AAV-2 Peptide Library Plasmids

An AAV-2 library based on the pMT187-0-3 backbone plasmid was designed to display a peptide with seven random amino acid residues within the VP capsid protein domain required for binding of AAV-2 to its natural receptor heparan sulfate (Wu et al., 2000; Xie et al., 2002); (FIG. 1A). The random peptide is flanked by two fixed amino acids, G and A (FIG. 1B). The diversity of the produced library plasmids was 1.1×10⁸ clones per library as determined from analysis of the unamplified plasmid library. The DNA of 20 clones was sequenced to verify that different random peptides are encoded in each clone (data not shown). Two of these clones with the insert LHLNVAL or AALAGSL, respectively, were amplified individually as well as the insertless backbone plasmid. The library backbone was designed such that the oligonucleotide insertion is required to shift the reading frame back to the original as in the wild-type cap gene. Consequently, the insertless library backbone plasmid pMT187-0-3 did not generate AAV particles, while the genomic and capsid titers of the particles obtained from clones LHLNVAL or AALAGSL were between 30 and 70% of the titers obtained from AAV-2 production using the pSub201 wild-type plasmid (FIG. 1C). Heparin binding of AAV generated from clones LHLNVAL or AALAGSL was reduced by 70-90% relative to the binding of wild-type AAV-2 (data not shown). These data suggest that mutations and insertions within the cap gene do not prevent efficient assembly of intact, genome containing AAV but diminish the natural AAV-2 tropism.

### EXAMPLE 3

### Production of the random AAV-2 display peptide library

After establishing the plasmid library, the AAV particle library production was performed in two additional steps (FIG. 2). We reasoned that direct transfer of the plasmid library into AAV producer cells by using conventional DNA transfection procedures may lead to uptake of more than one library plasmid per producer cell with subsequent production of chimeric capsids displaying more than a single type of a peptide insert. In that case the packaged capsid gene would not encode the capsid mutants displayed on the viral surface, impeding the selection process and subsequent identification of selected capsid mutants. To avoid this problem, the library containing mutant cap genes were packaged into capsids made up partially of wild-type VP protein by cotransfecting wild-type rep-cap plasmids (pXX2) lacking the ITRs required for encapsidation (Li et al., Journal of Virology 71 (1997), 5236-5243) along with the AAV plasmid library. The resulting AAV particles were termed “AAV library transfer shuttles”. After production, the genomic and replicative titers were determined to show that the shuttle production resulted in high titer preparations (Table 1). Shuttle particles bound heparin at a level of approximately 60% of wild-type AAV-2 (Table 2).

TABLE 1
Characterization of AAV library transfer shuttles
and random AAV display peptide libraries

| | | |
|---|---|---|
| | | replicative |
| genomic titer | capsid titer | titer |
| [mL⁻¹] | [mL⁻¹] | [mL⁻¹] |

AAV library transfer shuttles

| | | | |
|---|---|---|---|
| preparation^{a} | | | |
| 2/02 | 1.6 × 10¹² | 2.6 × 10¹² | 5 × 10⁸ |
| 3/02 | 1.8 × 10¹² | 1.1 × 10¹² | 10⁸ |

Random AAV display peptide libraries

| | | | |
|---|---|---|---|
| preparation^{b} | | | |
| 2/02-Lib | 5.9 × 10⁹ | 5.8 × 10⁹ | 10⁶ |
| 3/02-Lib | 6.5 × 10¹⁰ | 2.6 × 10¹⁰ | 10⁷ |

The genomic, capsid, and replicalive titers were determined as described (Grimm et al., 1999).
^{a}two shuttle preparations were made independently: 2/02 and 3/02.
^{b}two library preparations were made independently, one derived from shuttle preparation 2/02 (2/02-Lib) and one from shuttle preparation 3/02 (3/02-Lib)

TABLE 2
Heparin binding or library components and selected clones
Derivatives of shuttle preparation 3/02

| prepa- | shuttle | Library | Single clones | | |
|---|---|---|---|---|---|
| ration | 3/02 | 3/02-Lib | NSSRDLG | NDVRAVS | NSSRFDS |
| heparin | 56% | 36% | 11% | 4% | 11% |
| binding | | | | | |

Derivatives of shuttle preparation 2/02

| | | | |
|---|---|---|---|
| prepa- | shuttle | Library | single clones |

| ration | 2/02 | 2/02-Lib | NSVSSIA | NSVSSAS |
|---|---|---|---|---|
| heparin | 59% | n.a.^{a} | 9% | 1% |
| binding | | | | |

Heparin binding of wild-type controls was 100%.
^{a}the low capsid titer of the library did not allow for conduction of a heparin binding assay.

293T cells were infected with AAV library transfer shuttles at a multiplicity of infection (MOI) of 1 and 5 replicative units per cell. This way, we estimated uptake and propagation of approximately 1-5 library genomes per cell. Cells were superinfected with wild-type adenovirus type 5 (Ad5), resulting in efficient production of a random AAV-2 display peptide library (Table 1). Heparin binding analyzed in one library preparation was reduced to 36% of wild-type AAV-2 (Table 2). These results indicate that it is possible to produce AAV libraries efficiently in a two-step system from AAV- encoding plasmids containing random oligonucleotide inserts.

### EXAMPLE 4

Selection of AAV library clones targeting primary human coronary artery endothelial cells Primary human endothelial cells are poorly susceptible to wild-type AAV-2 transduction (Nicklin et al., 2001; Pajusola et al., Journal of Virology 76 (2002), 11530-11540). To select for AAV capsids that allow higher efficiency transduction of these cells, 1.2×10⁶ primary coronary artery endothelial cells were infected with the AAV library at an MOI of 100-400 capsids per cell. The low replicative titer of the initial libraries on coronary endothelial cells (10³/mL for 3/02-Lib and 10⁴/mL for 3/02-Lib) indicated a low number of clones within the AAV library displaying peptides that allow AAV binding and entry into coronary endothelial cells. Thus, we reasoned that the actual infection rate should not exceed one particle/cell in this setting. Cells were superinfected with Ad5, allowing for amplification of internalized AAV library clones. Amplified AAV were recovered and subjected to two more rounds of selection to enrich for AAV particles that bind to, are internalized by, and replicate within coronary artery endothelial cells. The DNA region containing the oligonucleotide insert of AAV particles recovered from all the stages of selection was amplified by PCR (FIG. 3). In addition to the expected 359 base pair band containing the insert, we also observed an additional, smaller band corresponding to the size of the wild-type capsid PCR product. This suggests that recombination events leading to packaging of wild-type cap genes into the shuttle capsids may have occurred during library transfer shuttle production. Interestingly, the wild-type band disappeared after the second selection round in library 3/02-Lib, indicating that library particles were internalized and therefore propagated more efficiently than wild-type AAV (FIG. 3). DNA sequencing of the subcloned PCR products revealed enrichment of peptide motifs after only two rounds of selection. None of the selected sequences was found in the initial, unselected AAV display peptide libraries (20 randomly isolated clones were sequenced; data not shown). At least three peptide motifs were enriched upon screening of the two library preparations (Tables 3 and 4). The most striking consensus motif was NSVRDL^{G}/_{S}, which was selected from library 3/02-Lib (Table 3). Another, entirely unrelated clone (PRSVTVP) was enriched from the same library (Table 3). The most prominent consensus motif derived from the 2/02-Lib selection was NSVSSX^{A}/ₛ (Table 4). It contained the tripeptide motif NSV which was also found in the 3/02-Lib selection consensus motif NSVRDL^{G}/ₛ. Homology of sequences also occurred in other clones selected from the two library preparations. PXSV contained within the 2/02-Lib-derived clone PTSVDAR was also part of the amino acid sequence of the PRSVPVP clone enriched from the 3/02 library (Tables 3 and 4). In addition, there were two clones sharing the motif SPLPSXS in round 1 of 2/02-Lib, however, these clones were not enriched in the subsequent round. The most frequently enriched clones from each round of selection were used for further analysis.

TABLE 3

Peptide sequences or AAV targeting primary
human coronary artery endothelial cells
derived from library preparation 3/02-Lib

| (A)Selection | | | |
|---|---|---|---|
| round | 1 | 2 | 3 |
| MOI^{a} | 400 | 1000 | 1000 |
| Sequence | NLVRGGD | NSSRDLG | NSSRDLG |
| | NLVRGGD | NSSRDLG | NSSRDLG |
| | NLVRGGD | NSSRDLG | NSSRDLG |
| | NLVRGGD | NSSRDLG | NSSRDLG |
| | NLVRGGD | NSSRDLG | NSSRDLG |
| | NTVHDLS | NSSRDLG | NSSRDLG |
| | NTVHDLS | NSSRDLG | NSSRDLG |
| | NTVHDLS | NSSRDLG | NSSRDLG |
| | NTVHDLS | NSSRDLG | NSSRDLG |
| | NSSRDLG | NSSRDLG | NSSRDLG |
| | NSSRDLG | NSSRDLG | NSSRDLG |
| | TGERGWA | NSVNDRS | NSSRDLG |
| | TGERGWA | NSVNDRS | NSSRDLG |
| | NVARVEM | NSVNDRS | NSSRDLG |
| | NVARVEM | NSVNDRS | NSSRDLG |
| | GFGDELH | NSVNDRS | TGERGWA |
| | NEVRDLS | NSVNDRS | |
| | NRVTDFP | NSVNDRS | |
| | NEVRLVS | NSSRPDS | |
| | NSVSFYE | NSSRFDS | |
| | NLVRGEG | NSSRFDS | |
| | DANYVRQ | NTVHDLS | |
| | NSAARET | NTVHDLS | |
| | NSTLPLS | (B) | |
| | NDVRAVS | | |

The NSVRDL^{S}/_{G} consensus motif is shown in blue, the enrichment of PRSVTVP is highlighted in orange.
^{a}MOI used for selection based on capsid titer.

TABLE 4

Peptide sequences or AAV targeting primary
human coronary artery endothelial cells
derived from library preparation 2/02-Lib

| (A)Selection | | |
|---|---|---|
| round | 1 | 2 |
| Sequence | NSVSSIA | NSVSSIA |
| | NSVSSIA | NSVSSIA |
| | NSVSSIA | NSVSSIA |
| | NSVSSIA | NSVSSIA |
| | NSVSSIA | NSVSSIA |
| | NSVSSIA | NSVSSIA |
| | NSVSSIA | NSVSSIA |
| | NSVSSIA | NSVSSIA |
| | NSVSSIA | NSVSSIA |
| | NSVSSAS | NSVSSIA |
| | NSVSSAS | NSVSSIA |
| | EMSTSRL | NSVSSIA |
| | ENSTSRL | NSVSSIA |
| | SPLPSPS | NSVSSIA |
| | SPLPSSS | NSVSSIA |
| | GQASAMG | NSVSSIA |
| | PSPNPSA | NSVSSIA |
| | PSTAGAS | NSVSSIA |
| | TLKGNVA | NSVSSIA |
| | TLKGNVA | NSVSSIA |
| | TLKGNVA | NSVSSAS |
| | TLKGNVA | NSVSSAS |
| | TLKGNVA | NSVSSAS |
| | PTSVDAR | NASRAEL |
| | LRLRYQP | NSTLAPM |

The NSVSSX^{S}/_{A} consensus motif is shown in green. The MOI used for selection was 100 capsid units cell.

### EXAMPLE 5

### Characterization of selected AAV clones targeting primary human coronary artery endothelial cells

Stocks of several AAV clones displaying a targeting peptide were produced and evaluated as follows: Analysis of heparin binding of five representative clones revealed a reduction to 1-11% relative to wild-type (Table 2). Infection and replication efficiency in primary coronary artery endothelial cells of targeted clones was compared to wild-type AAV-2 and a randomly selected control clone (LPQARSH) of an unselected AAV library. All selected clones showed replicative titers 10- to 630-fold higher than those of wild-type AAV-2 (FIG. 4A).

To validate the targeting effect of the selected peptides for AAV-mediated gene transfer, endothelial cells were transduced with targeted and untargeted vectors harboring a luciferase reporter gene. Gene transfer was enhanced 4- to 40-fold compared to AAV vectors carrying wild-type capsids (FIG. 4B). To evaluate cell type specificity of the selected capsid mutants, transduction efficiency was also investigated in a non-endothelial control cell line (HeLa). Transduction of HeLa cells resulted in 5- to 460-fold lower reporter activities compared to the vector carrying a wild-type capsid protein (FIG. 4C). These data suggest that the selected peptides within the library capsids target AAV vectors to human primary coronary artery endothelial cells and that the targeted receptors may not be ubiquitously expressed or activated in non-endothelial cells.

1. A method of producing a repertoire of random peptides on the surface of AAV particles, which method comprises:
expressing in a recombinant host cell nucleic acids encoding a diverse population of peptides, wherein each peptide is expressed as a fusion with an AAV capsid protein of an AAV particle which displays at its surface said diverse population of peptides, said AAV particle having the ability to replicate provided by genetic information packaged therewith and a helper vector providing the adenovirus helper functions.

2. The method of claim 1, wherein said AAV particles are AAV-2 particles.

3. The method of claim 2, wherein said capsid protein is VP1, VP2 or VP3 protein.

4. The method of claim 3, wherein oligonucleotides coding for said peptides are inserted at nucleotide position 3967 of the wild-type AAV-2 genome.

5. The method of claim 1, further comprising:
(a) transfecting host cells with (i) the AAV library genomes obtainable by the method of claim 4, (ii) a plasmid encoding the wild-type cap protein without ITRs and (iii) a helper vector providing the helper functions for AAV production;
(b) isolating the AAV library transfer shuttle vectors obtained from step (a);
(c) infecting host cells with the AAV library transfer shuttle vectors of step (b) and virus; and
(d) lysing and purifying the AAV capsids obtained in step (c).

6. The method of claim 5, wherein the virus in step (c) is adenovirus, HSV1, baculovirus or plasmids with viral helper genome.

7. The method of claim 6, wherein the adenovirus is Ad5 or Ad2.

8. The method of claim 1, wherein the host cell is a 293T cell.

9. A peptide library comprising a repertoire of random peptides on the surface of AAV particles which is obtainable by the method of claim 1.

10. A peptide library comprising a repertoire of random peptides on the surface of AAV particles which is obtainable by the method of claim 5.

11. A method of selecting a gene therapy vector specific for a desired cell type, comprising the following steps:
(a) infecting said desired cell type with a peptide library according to claim 9; and
(b) harvesting AAV library particles or PCR amplified viral DNA from the supernatant and/or cell lysates.

12. A method of selecting a gene therapy vector specific for a desired cell type, comprising the following steps:
(a) infecting said desired cell type with a peptide library according to claim 10; and
(b) harvesting AAV library particles or PCR amplified viral DNA from the supernatant and/or cell lysates.

13. The method of claim 12, wherein the desired cell type are human coronary artery endothelial cells.

14. An AAV vector targeting a primary human coronary artery endothelial cell, which is obtainable by the method of claim 13.

15. An AAV vector targeting a primary human coronary artery endothelial cell expressing a peptide as a fusion with a capsid protein, wherein said capsid protein comprises an amino acid sequence selected from the group consisting of the amino acid sequences of Table 3 or 4.

16. A method of gene therapy comprising the administration of an effective amount of a gene therapy AAV vector of claim 14.

17. A method of gene therapy comprising the administration of an effective amount of a gene therapy AAV vector of claim 15.

18. A method of treatment of a disease associated with a dysfunction of primary human coronary artery endothelial cells comprising the administration of an effective amount of a gene therapy AAV vector of claim 14.

19. A method of treatment of a disease associated with a dysfunction of primary human coronary artery endothelial cells comprising the administration of an effective amount of a gene therapy AAV vector of claim 15.

20. The method of claim 17, wherein said disease is coronary heart disease.

21. A peptide comprising an amino acid sequence selected from the group consisting of the amino acid sequences of Table 3 or 4.
